(19) United States
(12) Patent Application Publication
(10) Pub. No.: US 2010/0261783 A1
(43) Pub. Date: Oct. 14, 2010

(54) CRUCIFEROUS 3H-1,2-DITHIOLE-3-THIONE (D3T) AND METHODS OF PROTECTING AGAINST CELL INJURY

(76) Inventors: Zhenquan Jai, Blacksburg, VA (US); Yunbo Li, Blacksburg, VA (US); Hara Misra, Blacksburg, VA (US); Hong Zhu, Blacksburg, VA (US); Bhaba Ranjan Misra, Blacksburg, VA (US)

Correspondence Address: LATIMER INTELLECTUAL PROPERTY LAW, LLP, P.O. BOX 711200, HERNDON, VA, 20171, US

(21) Appl. No.: 12/709,442

(22) Filed: Feb. 19, 2010

Related U.S. Application Data
(60) Provisional application No. 61/153,774, filed on Feb. 19, 2009.

(51) Int. Cl. A61K 31/385; A61P 25/00; A61P 25/28; A61P 25/16

(52) U.S. Cl. 514/441

(57) ABSTRACT

Astrocytes possess important roles in maintaining normal brain function and providing trophic support to the neurons. They also suffer a range of toxic insults, being a chief target of prooxidants such as 1-methyl-4-phenyl-1,2,3,6-tetrahydropyridine (MPTP), 1-methyl-4-phenylpyridinium (MPP+), 6-hydroxydopamine (6-OHDA), 4-hydroxy-2-nonenal (HNE), and acrolein. Recently, we have observed that the cellular antioxidants and phase 2 enzymes can be upregulated by 3H-1,2-dithiole-3-thione (D3T), a nutraceutical found in cruciferous vegetables, against many prooxidants in human neuroblastoma cell lines (SH-SY5Y). However, the regulation of the above cellular factors by D3T in astrocytes and their role in ameliorating the neurotoxic effects of the above neurotoxins have not been investigated. In this study, we show that incubation of human primary astrocytes with micromolar concentrations (5-100 IM) of D3T for 24 h resulted in significant increases in the levels of reduced glutathione (GSH), glutathione reductase (GR), and the phase 2 enzyme NAD(P)H:quinone oxidoreductase 1 (NQO1). D3T treatment also caused time-dependent increases in mRNA expression of the gamma-glutamylcysteine ligase catalytic subunit (GCLC), GR, and of NQO1 in these cells. Pretreatment of astrocytes with D3T was found to afford remarkable protection against the neurocytotoxicity elicited by MPTP, MPP+, 6-OHDA, HNE and acrolein. Taken together, this study demonstrates for the first time that in human astrocytes, the cruciferous nutraceutical D3T potently induces the cellular GSH system and the phase 2 enzyme NQO1, which is accompanied by dramatically increased resistance of these cells to the damage induced by various neurotoxicants. The results of this study may have important implications for the development of novel neuroprotective strategies.

### CROSS-REFERENCE TO RELATED APPLICATIONS

This application relies on the disclosure of and claims the benefit of the filing date of U.S. Provisional Application No. 61/153,774, filed Feb. 19, 2009, the disclosure of which is incorporated by reference herein in its entirety.

### STATEMENT OF GOVERNMENT INTEREST

Supported in part by NIH Grant RO1HL071190 and a grant from the Harvey Peters Research Center Foundation and as such the U.S. government may have certain rights in the invention.

### BACKGROUND OF THE INVENTION

1. Field of the Invention

The present invention relates to the field of nutraceuticals.

2. Description of the Related Art

Astrocytes possess important roles in maintaining normal brain function and providing trophic support to the neurons. They are also chief target by a range of toxic insults of prooxidant neurotoxins including 1-methyl-4-phenyl-1,2,3,6-tetrahydropyridine (MPTP), 1-methyl-4-phenylpyridinium (MPP+), 6-hydroxydopamine (6-OHDA), 4-hydroxy-2-nonenal (HNE) and acrolein.

It has been observed that 3H-1,2-dithiole-3-thione (D3T)-pretreatment of SH-SY5Y cells (and induction of GSH, NQO1, and mitochondrial GSH) led to significant protection against the cytotoxicity elicited by the neurotoxicants dopamine, 6-hydroxydopamine, 4-hydroxy-2-nonenal, and H₂O₂. See “Potent induction of total cellular GSH and NQO1 as well as mitochondrial GSH by 3H-1,2-dithiole-3-thione in SH-SY5Y neuroblastoma cells and primary human neurons: Protection against neurocytotoxicity elicited by dopamine, 6-hydroxydopamine, 4-hydroxy-2-nonenal, or hydrogen peroxide,” Zhenquan Jai, et al., Brain Res., 1197 (2008) 159-169, the disclosure of which is herein incorporated by reference in its entirety.

It has been demonstrated that D3T potently upregulated many antioxidants and phase 2 enzymes in human cardiomyocytes, which was accompanied by increased resistance to oxidative/electrophilic stress and doxorubicin toxicity. See “Cruciferous Dithiolethione-Mediated Coordinated Induction of Total Cellular and Mitochondrial Antioxidants and Phase 2 Enzymes in Human Primary Cardiomyocytes: Cytoprotection Against Oxidative/Electrophilic Stress and Doxorubicin Toxicity,” Hong Zhu, et al., Exp. Biol. Med. 2009;234, 418-429, the disclosure of which is herein incorporated by reference in its entirety.

### SUMMARY OF THE INVENTION

The present invention provides a unique nutraceutical found in the daily vegetable for protection of human astrocytes against free radicals and electrophilic attacks by way of enhancing endogenous antioxidants and phase 2 enzymes; a unique mechanism for protection of neuronal cells against free radicals and electrophilic attacks by way of enhancing endogenous antioxidants and phase 2 enzymes; and a unique mechanism for myocardial protection by way of enhancing endogenous antioxidant defenses.

### DETAILED DESCRIPTION OF VARIOUS EMBODIMENTS OF THE INVENTION

Reference will now be made in detail to various exemplary embodiments of the invention. The following detailed description is presented for the purpose of describing certain embodiments in detail and is, thus, not to be considered as limiting the invention to the embodiments described. Rather, the true scope of the invention is defined by the claims.

Embodiments of the present invention include the novel cruciferous nutrichemical 3H-1,2-dithiole-3-thione (D3T), which is capable of inducing cellular antioxidants and phase 2 enzymes in human neuronal cells (e.g., neuroblastoma SH-SY5Y cells). See “Upregulation of Cellular Gluththione 3H-1,2-dithiole-3-thione (D3T) as a Possible Treatment Strategy for Protecting Against Acrolein-Induced neurocytotoxicity,” Zhenquan Jai et al., (Accepted Nov. 21, 2008), NeuroToxicology, the disclosure of which is herein incorporated by reference in its entirety.

This nutrichemical also protected against acrolein-induced neurotoxicity in these cells. Acrolein, an unsaturated aldehydic product of lipid peroxidation (free radical-mediated lipid breakdown product), has been implicated in the pathogenesis of various neurodegenerative disorders including Alzheimer's and Parkinson's diseases. However, whether chemical upregulation of the cellular antioxidants and aldehyde-detoxification factors affords protection against acrolein toxicity in neuronal cells has not been investigated. We have recently found that treatment of cells with D3T resulted in a marked concentration- and time-dependent induction of glutathione (GSH) and also led to increased protein and mRNA expression of gamma-glutamylcysteine ligase (GCL), the key enzyme in GSH biosynthesis, and afforded a dramatic protection against acrolein-induced cytotoxicity in human neuroblastoma SH-SY5Y cells providing a possible treatment/preventive strategy for neurodegenerative diseases including Alzheimer's and Parkinson's Diseases.

Embodiments of the invention include See “Cruciferous Nutraceutical 3H-1,2-dithiole-3-thione Protects Human Primary Astrocytes Against Neurocytotoxicity Elicited by MPTP, MPP+, 6-OHDA, HNE and Acrolein,” Zhenquan Jai et al., (published on-line May 1, 2009), Neurochem Res (2009) 34:1924-34, the disclosure of which is herein incorporated by reference in its entirety.

For example, 3H-1,2-dithiole-3-thione (D3T), a cruciferous nutraceutical found in cruciferous vegetable is able to induce a number of cellular antioxidants including glutathione (GSH) and phase 2 enzymes in human primary astrocytes, can result in protection of oxidative and electrophilic cell injury. Since oxidative stress-induced loss of astrocyte glutathione could directly compromise astrocyte viability and therefore indirectly affect neuronal survival, the potent induction of cellular glutathione and other antioxidant enzymes by the cruciferous nutraceutical D3T will contribute to the development of novel strategies against neurodegenerative disorders including Alzheimer's and Parkinson's Diseases.

Embodiments of the invention include 3H-1,2-dithiole-3-thione (D3T) to induce cellular antioxidant defenses and protect against free radical injury in normal human cardiomyocytes. D3T potently induced a series of antioxidants in primary human cardiomyocytes. Even further, D3T potently induced antioxidants in mitochondria of the human cardiomyocytes. D3T conferred nearly complete protection of the cardiomyocytes from injury caused by a wide range of free radicals and electrophiles implicated in human cardiac diseases, such as myocardial infarction and heart failure D3T also potently protected human cardiomyocytes from injury elicited by doxorubicin, a widely used anticancer drug with significant adverse effects on heart. These novel uses of the cruciferous D3T may provide a novel approach for intervention of human myocardial injury from ischemia as well as by doxorubicin.

The present invention has been described with reference to particular embodiments having various features. It will be apparent to those skilled in the art that various modifications and variations can be made in the practice of the present invention without departing from the scope or spirit of the invention. One skilled in the art will recognize that these features may be used singularly or in any combination based on the requirements and specifications of a given application or design. Other embodiments of the invention will be apparent to those skilled in the art from consideration of the specification and practice of the invention. The description of the invention provided is merely exemplary in nature and, thus, variations that do not depart from the essence of the invention are intended to be within the scope of the invention.

1. A method of protecting against acrolein-induced neurocytotoxicity comprising treating brain cells with D3T prior to exposure to acrolein.

2. The method of claim 1, wherein the brain cells are SH-SY5Y cells, the amount of D3T is in the range of about 10-100 μM.

3. The method of claim 2, which is a method of protecting against the onset or further development of neurodegenerative disorders.

4. The method of claim 3, wherein the neurodegenerative disorder is chosen from Alzheimer's and Parkinson's Diseases.
